SEQUENCE LISTING (12 sequences)

SEQ ID NO: 1 - DNA, 2628 bp, Artificial Sequence
  It is synthesized
atggctctgcctgtgaccgccctgctgctgcctctggctctgctgctgcacgccgctcggcctgacatcgttttgacacaatctcctgcgtcattggccatgagtctcgggaagcgcgcaacaatatcctgtcgcgccagtgaatctgtgtctgtgataggagcgcacttgatccattggtatcagcagaaacctggacaacctcccaagctgctcatctacctcgccagtaaccttgaaacaggagtacctgctcggttttcaggttccgggtcagggacggatttcactttgactatcgacccagttgaggaagacgacgtagccatatatagctgcctgcagtctcggatcttcccgcgcacgttcgggggaggaactaagctggagattaagggcggcgggggttctggtggcggcggcagcggcggtggaggatcacaaatccaactggttcagtccggtccagaactgaaaaagccgggggagacggtgaaaatctcctgtaaggcctcaggttataccttcaccgattacagcatcaattgggtaaagcgggctccagggaaaggtctgaaatggatgggttggatcaacacagaaacccgagaaccagcctatgcttacgactttcgaggtcgattcgctttttccttggaaacttccgcaagcacagcctatctgcaaatcaacaatctcaagtacgaagatacggccacgtatttttgtgccctggattacagctatgcaatggattactggggtcaggggacgtctgttacagtttctagtactacaactccagcacccagaccccctacacctgctccaactatcgcaagtcagcccctgtcactgcgccctgaagcctgtcgccctgctgccgggggagctgtgcatactcggggactggactttgcctgtgatatctacatctgggcgcccttggccgggacttgtggggtccttctcctgtcactggttatcaccctttactgcaggttcagtgtcgtgaagagaggccggaagaagctgctgtacatcttcaagcagcctttcatgaggcccgtgcagactacccaggaggaagatggatgcagctgtagattccctgaagaggaggaaggaggctgtgagctgagagtgaagttctcccgaagcgcagatgccccagcctatcagcagggacagaatcagctgtacaacgagctgaacctgggaagacgggaggaatacgatgtgctggacaaaaggcggggcagagatcctgagatgggcggcaaaccaagacggaagaacccccaggaaggtctgtataatgagctgcagaaagacaagatggctgaggcctactcagaaatcgggatgaagggcgaaagaaggagaggaaaaggccacgacggactgtaccaggggctgagtacagcaacaaaagacacctatgacgctctgcacatgcaggctctgccaccaagacgagctaaacgaggctcaggcgcgacgaactttagtttgctgaagcaagctggggatgtagaggaaaatccgggtcccatgttgctccttgtgacgagcctcctgctctgcgagctgccccatccagccttcctcctcatcccgcggaaggtgtgcaatggcataggcattggcgagtttaaagattctctgagcataaatgctacgaatattaagcatttcaagaattgtacttctattagtggcgacctccatattcttccggttgccttcaggggtgactctttcacccacacacctccattggatccacaagaacttgacatcctgaagacggttaaagagattacaggcttcctccttatccaagcgtggcccgagaacagaacggacttgcacgcctttgagaacctcgaaataatacggggtcggacgaagcaacacggccaatttagccttgcggttgttagtctgaacattacttctctcggccttcgctctttgaaagaaatcagcgacggagatgtcatcattagtggaaacaagaacctgtgctacgcgaacacaatcaactggaagaagctcttcggtacttcaggccaaaagacaaagattattagtaacagaggagagaatagctgtaaggctaccggacaagtttgtcacgccttgtgtagtccagagggttgctggggaccggaaccaagggattgcgtcagttgccggaacgtgagtcgcggacgcgagtgtgtggataagtgcaatcttctggaaggggaaccgcgagagtttgtagaaaattccgaatgtatacagtgtcatcccgagtgtcttccacaagcaatgaatatcacatgtacagggaggggtcctgataactgtatccaatgtgcacactacatagatggtcctcactgtgtaaagacgtgccccgccggagtaatgggtgaaaacaacaccctcgtgtggaagtacgccgatgccgggcatgtctgtcatttgtgtcatcccaactgcacatatggctgtaccggtcctggattggagggctgtccaacaaacgggccgaaaataccgagtatcgcaacaggcatggtgggagcacttttgcttctcctcgttgtcgccctgggcatcggcttgttcatgtga

SEQ ID NO: 2 - protein, 874 aa, Artificial Sequence
  It is synthesized.
MALPVTALLLPLALLLHAARPDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 3 - DNA, 21 bp, Artificial Sequence
  It is synthesized.
agcatcgttctgtgttgtctc

SEQ ID NO: 4 - DNA, 22 bp, Artificial Sequence
  It is synthesized.
tgtttgtcttgtggcaatacac

SEQ ID NO: 5 - DNA, 21 bp, Artificial Sequence
  It is synthesized.
agcatcgttctgtgttgtctc

SEQ ID NO: 6 - DNA, 22 bp, Artificial Sequence
  It is synthesized.
tgtttgtcttgtggcaatacac

SEQ ID NO: 7 - protein, 21 aa, Artificial Sequence
  It is synthesized.
GSGGGGSGGGGSGGGLGSTEF

SEQ ID NO: 8 - protein, 4 aa, Artificial Sequence
  It is synthesized.
GGGS

SEQ ID NO: 9 - protein, 5 aa, Artificial Sequence
  It is synthesized.
GGGGS

SEQ ID NO: 10 - protein, 5 aa, Artificial Sequence
  It is synthesized.
SSSSG

SEQ ID NO: 11 - protein, 5 aa, Artificial Sequence
  It is synthesized.
GSGSA

SEQ ID NO: 12 - protein, 5 aa, Artificial Sequence
  It is synthesized.
GGSGG

(19) United States
(12) Patent Application Publication
(10) Pub. No.: US 2021/0221903 A1
(43) Pub. Date: Jul. 22, 2021

(54) BCMA-TARGETING CHIMERIC ANTIGEN RECEPTOR AND USES THEREOF

(71) Applicant: HRAIN BIOTECHNOLOGY CO., LTD., Shanghai (CN)

(72) Inventors: Yarong LIU, Shanghai (CN); Feng HE, Shanghai (CN); Haiying WANG, Shanghai (CN); Zixiao SHI, Shanghai (CN)

(21) Appl. No.: 17/259,868

(22) PCT Filed: Aug. 14, 2018
(86) PCT No.: PCT/CN2018/100369

§ 371 Date: Jan. 12, 2021

(51) Int. Cl. C07K 16/28; C07K 14/705; C07K 14/725; C07K 14/71; A61K 35/17; C12N 15/86; A61P 35/00

(52) U.S. Cl. CPC C07K 16/2878; C07K 14/70517; C07K 14/70578; C07K 14/7051; C07K 14/71; A61K 35/17; C12N 2740/10043; C07K 14/70521; A61P 35/00; C07K 2319/03; C07K 2319/33; C07K 2319/02; C07K 2317/622; C12N 15/86

(57) ABSTRACT

The disclosure relates to a BCMA-targeting chimeric antigen receptor and uses thereof. Specifically, the disclosure provides a polynucleotide sequence selected from the group consisting of: (1) a polynucleotide sequence comprising, linked in sequence, a sequence encoding an anti-BCMA single chain antibody, a sequence encoding the hinge region of human CD8α, a sequence encoding the transmembrane region of human CD8, a sequence encoding the intracellular domain of human 41BB, a sequence encoding the intracellular domain of human CD3ζ and optionally a sequence encoding a fragment of EGFR comprising the extracellular domain III and the extracellular domain IV; and (2) a complementary sequence of the polynucleotide sequence of (1). The disclosure further provides corresponding fusion proteins and vectors comprising the coding sequence, as well as uses of the fusion proteins, coding sequences and vectors.

### TECHNICAL FIELD

The present disclosure generally relates to chimeric antigen receptors, particularly BCMA-targeting chimeric antigen receptors and uses thereof.

### BACKGROUND

Multiple myeloma is a malignant plasmacyte disease characterized in malignant clonal proliferation of bone marrow plasmacytes secreting monoclonal immunoglobulins or fragments thereof (M protein), which causes damages to target organs or tissues such as bones and kidneys, with clinical manifestations like ostealgia, anemia, renal insufficiency and infection [Multiple myeloma. N Engl J Med, 2011. 364(11): p. 1046-60]. Multiple myeloma is now the second leading malignant hematologic tumor that accounts for 10% of hematological malignancies, which is more common in male with incidence increasing with age but trending younger lately [Siegel, R., et al., Cancer statistics, 2014. CA Cancer J Clin, 2014. 64(1): p. 9-29.].

B-cell maturation antigen (BCMA), also known as CD269, consists of 184 amino acid residues, which comprises an intracellular domain comprising 80 amino acid residues and a very short extracellular domain comprising a single carbohydrate-recognition domain as a B-cell surface molecule. BCMA is a type I transmembrane signaling protein lacking signal peptide and is a member of the family of tumor necrosis factor receptors (TNFRs). It is capable of binding to the B cell activation factor (BAFF) and the proliferation-induced ligand (APRIL) [Tumor necrosis factor family ligand-receptor binding. Curr Opin Struct Biol, 2004. 14(2): p. 154-60.]. In normal tissues, BCMA is surface expressed on mature B cells and plasmacytes. BCMA gene-knocked out mice exhibit normal immune system functionality, normal spleen structure, normal B lymphocyte development, except for decreased level of plasmacytes. This demonstrates that BCMA plays an important role in survival of plasmacytes, wherein the mechanism of action mainly involves the BCMA's binding to BAFF, upregulating of anti-apoptosis genes like Bcl-2, Mcl-1, Bclw, etc., maintenance of cell growth [BCMA is essential for the survival of long-lived bone marrow plasma cells. J Exp Med, 2004. 199(1): p. 91-8.]. Similarly, the action also plays an important role in promoting malignant proliferation in myeloma cells [BAFF and APRIL protect myeloma cells from apoptosis induced by interleukin 6 deprivation and dexamethasone. Blood, 2004. 103(8): p. 3148-57.]. It has been shown that BCMA is ubiquitously expressed in multiple myeloma cell lines, which has been confirmed by the detection in multiple myeloma patients [Expression of BCMA, TACI, and BAFF-R in multiple myeloma: a mechanism for growth and survival. Blood, 2004. 103(2): p. 689-94.]. Kochenderfer et al., on basis of the previously reported results, further explored the expression profile of BCMA using multiple techniques including Q-PCR, Flow Cytometry and immunohistochemistry, and concluded that BCMA is not expressed on normal human tissues except for mature B cells and plasmacytes, and is not expressed on CD34+ hematopoietic cells [B-cell maturation antigen is a promising target for adoptive T-cell therapy of multiple myeloma. Clin Cancer Res, 2013. 19(8): p. 2048-60.]. In view of the high similar expression profiles between BCMA and CD19, along with the progress in anti-CD19 CAR T-cell therapy, we recognize that BCMA is a potential target of CAR-T-cells for use in cellular immunotherapy of multiple myeloma.

Chimeric Antigen Receptor-T-cells (CAR-T-cells) are the T-cells that are genetically modified to be capable of non-MHC-restricted antigen recognition and sustained activation to proliferation. At the 2012 Annual Meeting of the International Society for Cellular Therapy, it was reported that in addition to surgery, radiation and chemotherapy, biological immune cell therapy has been recognized as the fourth anti-tumor therapy, which will become an essential measure of anti-tumor therapy in future. CAR-T-cell autologous infusion is presently the anti-tumor immune therapy with the most definite efficacy. Extensive studies have shown that CAR-T-cells can effectively recognize tumor antigens, elicit specific anti-tumor immune response and significantly improve patients' well-being.

Chimeric antigen receptor (CAR) is core to CAR-T, which render T-cells the HLA-independent recognition of tumor antigens. This expands the spectrum of targets for CAR-modified T-cells, compared to native T-cell surface receptors (TCR). A design of CAR may basically include a tumor-associated antigen (TAA) binding domain, which is usually derived from a scFV fragment of the antigen-binding domains of a monoclonal antibody, an extracellular hinge domain, a transmembrane region and an intracellular signaling domain. Selection of the target antigen is crux for specificity and efficacy of the CAR, as well as safety of the genetically modified T-cells per se.

The chimeric Antigen Receptor-T-cell (CAR-T) technology has been developing through four generations.

CAR-T-cells of the first generation are composed of an extracellular binding domain-single chain antibody (single chain fragment variable, scFV), a transmembrane region (TM) and an intracellular signaling domain-immunoreceptor tyrosine based activation motif (ITAM), wherein said components of the chimeric antigen receptor are linked in the format of scFv-TM-CD3ζ. Though CARs of the first generation were observed with certain degree of specific cytotoxicity, a review of clinical trials in 2006 revealed that the efficacy in therapy is barely satisfactory. This may be attributable to the fact that CAR-T-cells of the first generation is cleared-off quickly in patients and the sustainability is so poor that the CAR-T-cells go to apoptosis before they even get into contact with a substantive amount of tumor cells. These CAR-T-cells are capable of eliciting an anti-tumor cytotoxicity with less cytokine secretion, but are not capable of eliciting a sustainable anti-tumor cytotoxicity due to their short lifetime in vivo (Chimeric NKG2D-modified T-cells inhibit systemic T-cell lymphoma growth in a manner involving multiple cytokines and cytotoxic pathways, Cancer Res 2007, 67(22): 11029-11036).

CAR-T-cells of the second generation include in the CAR design an optimized signaling domain for T-cell activation, which is continuously a topic of great interests of R&D. Full activation of T-cell requires actions of the two-signal pathways and cytokines, wherein the first signal is specific and is triggered by TCR's recognition of the antigen-MHC complex on surface of antigen presenting cells, while the second signal is a co-stimulatory signal. Development of the 2^{nd} generation CAR can be traced back to 1998 (J Immunol. 1998; 161(6): 2791-7). The 2^{nd} generation incorporated a co-stimulatory signal molecule into the intracellular signal peptide region, i.e., assembling the co-stimulatory signal into CAR, so as to improve the activation signaling to CAR-T-cells. These CARs, upon recognizing tumor cells, activate both the co-stimulatory molecule and the intracellular signal at the same time, which provides a dual-activation to significantly increase proliferation, secretion and anti-tumor effect of the T-cells. CD28 is the first T-cell co-stimulatory signal receptor that was studied, which is found capable of binding B7 family members on target cells. Co-stimulation via CD28 promotes T-cell proliferation, IL-2 synthesis and expression, as well as the resistance to apoptosis in T-cells. Since then, there came more co-stimulatory molecules, including CD134(OX40), 41BB(4-1BB), etc., which provide T-cells with improvements in activities including cytotoxicity, proliferation, sustained T-cell response, prolonged T-cell survival, etc. Later, these 2^{nd} generation CARs exhibited surprising effects in clinical trials. Since 2010, the 2^{nd} generation CARs have been repeatedly reported with clinical trials provoking great repercussion, especially in recurrent and refractory ALL patients, where they provided a complete response rate over 90%.

CARs of the third generation incorporated into the signal region two or more co-stimulatory molecules, which provide T-cells with sustained activation and proliferation, sustained cytokine secretion, and improved T-cell killing of tumor cells. These CARs of the new generation provide enhanced anti-tumor response (Mol Thu., 2005, 12(5): 933-941). As the most representative event, professor Carl June from U Pen added a 41BB stimulator under the action of the CD28 stimulator.

CAR-T-cells of the fourth generation incorporated cytokines or co-stimulatory ligands. For example, a CAR of the 4^{th} generation is capable of producing IL-12 and modulating immunological niche to increase T cell activation and, at the same time, to activate the native immune cells to effect depletion of cancer cells negative for the target antigen, whereby renders a bidirectional regulation (TRUCKS: the fourth generation of CARs, Expert Opin Biol Ther., 2015; 15(8): 1145-54).

A big advantage of CAR-T-cells is that upon infusion as a pharmaceutically active ingredient, the T-cell balance, memory formation and antigen-driven expansion will be regulated by the physiological mechanisms. Nevertheless, this therapy is not established yet. Off-target T-cells may attack other tissues, or the expansion may go rampant in excess of the need for therapy. Since CAR-T-cell has been included into the range of standard treatments, designs of patient- or medicine-controllable on-or-off mechanism of CAR-T-cell regulation will be of great value. In technology, off-mechanisms are more applicable to T-cells. One example is the iCas9 system that is undergoing clinical studies. In cells expressing iCas9, dimerization of iCas9 precursors can be induced and apoptosis pathway activated using small molecules to cause cell depletion. The small molecule AP1903 has been used in graft-versus-host diseases to induce iCas9 dimers and T-cell depletion, which indicates feasibility of this approach (Making Better Chimeric Antigen Receptors for Adoptive T-cell Therapy, Clin Cancer Res; 22(8), Apr. 15, 2016).

Additionally, depletory antibodies already used in clinic may also be used. In one instance, CAR-T-cells may be modified to co-express proteins against which these antibodies are directed, like tEGFR, which enables depletion of these CAR-T-cells using the antibodies after induction of the therapeutic toxicity or upon completion of the therapy (Rational development and characterization of humanized anti-EGFR variant III chimeric antigen receptor T-cells for glioblastoma, Sci Transl Med 2015; 7: 275ra22).

September 2015, N Engl J Med published the success by Carl June and his team in treating a recurrent refractory multiple myeloma (MM) using a CD19-targeted CAR-T-cell therapy [Chimeric Antigen Receptor T Cells against CD19 for Multiple Myeloma. N Engl J Med, 2015. 373(11): p. 1040-7.]. Multiple myeloma as a B-cell tumor does not normally express CD19, and therefore, CD19 is normally not an immunotherapy target for multiple myeloma. It has been reported that very few resistant and recurrent multiple myeloma clones have the B-cell phenotype (i.e., CD19 positive). With the knowledge that BCMA is a useful target of CAR T-cells, Dr. Kochenderfer and his colleagues from the National Cancer Institute constructed anti-BCMA CAR T-cells, which were observed with specific recognition of BCMA, substantial expansion upon activation by BCMA, cytokine secretion and killing effect in preclinical studies, and were observed with an anti-tumor response in a neoplastic model in mouse [B-cell maturation antigen is a promising target for adoptive T-cell therapy of multiple myeloma. Clin Cancer Res, 2013. 19(8): p. 2048-60.]. In 2014, the National Cancer Institute started a phase I clinical trial of an anti-BCMA CAR T-cell therapy of multiple myeloma to assess efficacy and safety of this therapy in multiple myeloma patients that are irresponsive to the current standard treatment (ClinicalTrials. gov Identifier: NCT02215967). Early December 2015, at the 57^{th} annual meeting of the American Society of Hematology, professor Syed Abbas Ali and his team from the oncology department of the National Cancer Institute reported a phase I clinical trial of a CAR-T-cell therapy in multiple myeloma patients. This trial enrolled twelve (12) refractory and recurrent multiple myeloma patients irresponsive to chemotherapies of the third-line and above, including some patients having myeloma cells≥50% in bone marrow. After receiving infusion of BCMA CAR-T-cells, a complete response is achieved in one (1) patient, partial response in three (3) and stable in the rest. This proved for the first time that anti-BCMA CAR-T-cell therapy is effective in multiple myeloma, without significant side effects. This trial is listed by ASH as one of the most influential clinical trails of the year (Late-Breaking Abstracts, Conference Summary No.: LAB-1). Recently, Abramson Cancer Center University of Pennsylvania has also registered a phase I clinical trial of anti-BCMA CAR T-cell therapy in multiple myeloma, which is undergoing intensive investigation and advancement (Clinical Trials. gov Identifier: NCT02546167).

### SUMMARY OF INVENTION

In the first aspect, the present disclosure provides a polynucleotide sequence selected from the group consisting of:

(1) a polynucleotide sequence comprising the followings linked in sequence: a sequence encoding an anti-BCMA single chain antibody, a sequence encoding the hinge region of human CD8α, a sequence encoding the transmembrane region of human CD8, a sequence encoding the intracellular domain of human 41BB, a sequence encoding the intracellular domain of human CD3ζ and optionally a sequence encoding a fragment of EGFR comprising the extracellular domain III and the extracellular domain IV; and

(2) a complementary sequence of the polynucleotide sequence of (1).

In one or more embodiments, the polynucleotide sequence further comprises in front of the sequence encoding the anti-BCMA single chain antibody a sequence encoding a signal peptide. In one or more embodiments, the signal peptide has the sequence of amino acids 1-21 as set forth in SEQ ID NO:2. In one or more embodiments, the anti-BCMA single chain antibody comprises a light chain variable region having the sequence of amino acids 22-132 as set forth in SEQ ID NO:2. In one or more embodiments, the anti-BCMA single chain antibody comprises a heavy chain variable region having the sequence of amino acids 148-264 as set forth in SEQ ID NO:2. In one or more embodiments, the hinge region of human CD8α has the sequence of amino acids 265-311 as set forth in SEQ ID NO:2. In one or more embodiments, the transmembrane region of human CD8 has the sequence of amino acids 312-333 as set forth in SEQ ID NO:2. In one or more embodiments, the intracellular domain of human 41BB has the sequence of amino acids 334-381 as set forth in SEQ ID NO:2. In one or more embodiments, the intracellular domain of human CD3ζ has the sequence of amino acids 382-492 as set forth in SEQ ID NO:2. In one or more embodiments, the fragment of EGFR comprises or consists of the extracellular domain III, the extracellular domain IV and the transmembrane region of EGFR. In one or more embodiments, the fragment of EGFR comprises or consists of the sequence of amino acids 310-646 of human EGFR. In one or more embodiments, the polynucleotide sequence further comprises a sequence encoding the signal peptide of the α chain of GM-CSF receptor, wherein the signal peptide of the α chain of GM-CSF receptor is positioned N-terminal to the fragment of EGFR. In one or more embodiments, the signal peptide of the α chain of GM-CSF receptor has the sequence of amino acids 518-539 as set forth in SEQ ID NO:2. In one or more embodiments, the polynucleotide sequence further comprises a sequence encoding a linker between the signal peptide of the α chain of GM-CSF receptor and the human CD3ζ intracellular domain. In one or more embodiments, the linker has the sequence of amino acids 493-517 as set forth in SEQ ID NO:2.

In one or more embodiments, the sequence encoding a signal peptide ahead of the sequence encoding an anti-BCMA single chain antibody has the sequence of nucleotides 1-63 as set forth in SEQ ID NO:1. In one or more embodiments, the sequence encoding the light chain variable region of the anti-BCMA single chain antibody has the sequence of nucleotides 64-396 as set forth in SEQ ID NO:1. In one or more embodiments, the sequence encoding the heavy chain variable region the anti-BCMA single chain antibody has the sequence of nucleotides 442-792 as set forth in SEQ ID NO:1. In one or more embodiments, the sequence encoding the hinge region of human CD8α has the sequence of nucleotides 793-933 as set forth in SEQ ID NO:1. In one or more embodiments, the sequence encoding the transmembrane region of human CD8 has the sequence of nucleotides 934-999 as set forth in SEQ ID NO:1. In one or more embodiments, the sequence encoding the intracellular domain of human 41BB has the sequence of nucleotides 1000-1143 as set forth in SEQ ID NO:1. In one or more embodiments, the sequence encoding the intracellular domain of human CD3ζ has the sequence of nucleotides 1144-1476 as set forth in SEQ ID NO:1. In one or more embodiments, the sequence encoding the linker between the signal peptide of the α chain of GM-CSF receptor and the intracellular domain of human CD3ζ has the sequence of nucleotides 1477-1554 as set forth in SEQ ID NO:1. In one or more embodiments, the sequence encoding the signal peptide of the a chain of GM-CSF receptor has the sequence of nucleotides 1555-1634 as set forth in SEQ ID NO:1. In one or more embodiments, the sequence encoding the fragment of EGFR has the sequence of nucleotides 1635-2628 as set forth in SEQ ID NO:1. In one or more embodiments, the polynucleotide sequence encodes the sequence of amino acids 22-492 as set forth in SEQ ID NO:2 or the sequence of amino acids 24-878 as set forth in SEQ ID NO:2 or the amino acid sequence of SEQ ID NO:2. In one or more embodiments, the polynucleotide sequence comprises or consists of the nucleotide sequence of SEQ ID NO:1, the sequence of nucleotides 1-1634 as set forth in SEQ ID NO:1, the sequence of nucleotides 64-1476 as set forth in SEQ ID NO:1 or the sequence of nucleotides 64-2628 as set forth in SEQ ID NO:1.

In the second aspect, the present disclosure provides a fusion protein selected from the group consisting of:

(1) a fusion protein comprising the followings linked in sequence: an anti-BCMA single chain antibody, the hinge region of human CD8α, the transmembrane region of human CD8, the intracellular domain of human 41BB and the intracellular domain human CD3ζ, as well as optionally a fragment of EGFR comprising the extracellular domain III and the extracellular domain IV; and

(2) a fusion protein derived from (1), comprising one or more substitution(s), deletion(s) or addition(s) in the amino acid sequence of (1) while retaining the activity of T-cell activation;

wherein, the anti-BCMA single chain antibody is preferably the anti-BCMA monoclonal antibody C11D5.3.

In one or more embodiments, the fusion protein further comprises a signal peptide N-terminal to the anti-BCMA single chain antibody. In one or more embodiments, the signal peptide has the sequence of amino acids 1-21 as set forth in SEQ ID NO:2. In one or more embodiments, the anti-BCMA single chain antibody comprises a light chain variable region having the sequence of amino acids 22-132 as set forth in SEQ ID NO:1. In one or more embodiments, the anti-BCMA single chain antibody comprises a heavy chain variable region having the sequence of amino acids 148-264 as set forth in SEQ ID NO:1. In one or more embodiments, the hinge region of human CD8α has the sequence of amino acids 265-311 as set forth in SEQ ID NO:1. In one or more embodiments, the transmembrane region of human CD8 has the sequence of amino acids 312-333 as set forth in SEQ ID NO:1. In one or more embodiments, the intracellular domain of human 41BB has the sequence of amino acids 334-381 as set forth in SEQ ID NO:1. In one or more embodiments, the intracellular domain of human CD3ζ has the sequence of amino acids 382-492 as set forth in SEQ ID NO:1. In one or more embodiments, the fragment of EGFR comprises or consists of the extracellular domain III, the extracellular domain IV and the transmembrane region of EGFR. In one or more embodiments, the fragment of EGFR comprises or consists of the sequence of amino acids 310-646 of human EGFR. In one or more embodiments, the fragment of EGFR has the sequence of amino acids 540-874 as set forth in SEQ ID NO:1. In one or more embodiments, the fusion protein further comprises the signal peptide of the α chain of GM-CSF receptor, which is positioned N-terminal to the fragment of EGFR. In one or more embodiments, the signal peptide of the α chain of GM-CSF receptor has the sequence of amino acids 518-539 as set forth in SEQ ID NO:2. In one or more embodiments, the fusion protein further comprises a linker between the signal peptide of the α chain of GM-CSF receptor and the human CD3ζ intracellular domain. In one or more embodiments, the linker has the sequence of amino acids 493-517 as set forth in SEQ ID NO:2. In one or more embodiments, the fusion protein has the sequence of amino acids 22-492 as set forth in SEQ ID NO:2, or the sequence of amino acids 22-646 as set forth in SEQ ID NO:2 or the amino acid sequence of SEQ ID NO:2.

In the third aspect, the present disclosure provides a nucleic acid construct comprising the polynucleotide sequence according to the present disclosure.

In one or more embodiments, the nucleic acid construct is a vector. In one or more embodiments, the nucleic acid construct is a retrovirus vector comprising an origin of replication, a 3′LTR, a 5′LTR and the polynucleotide sequence of the present disclosure, as well as an optional selection marker.

In the fourth aspect, the present disclosure provides a retrovirus which comprises the nucleic acid construct, preferably the vector, more preferably the retrovirus vector according to the present disclosure.

In the fifth aspect, the present disclosure provides a genetically modified T-cell which comprises the polynucleotide sequence or the nucleic acid construct according to the present disclosure, or is infected with the retrovirus according to the present disclosure, or stably expresses the fusion protein and optionally the fragment of EGFR comprising the extracellular domain III, the extracellular domain IV and optionally the transmembrane region of the receptor.

In the sixth aspect, the present disclosure provides a pharmaceutical composition comprising the genetically modified T-cell according to the present disclosure.

In the seventh aspect, the present disclosure provides use of the polynucleotide sequence, the fusion protein, the nucleic acid construct or the retrovirus according to the present disclosure for producing activated T-cells.

In the eighth aspect, the present disclosure provides use of the polynucleotide sequence, the fusion protein, the nucleic acid construct, the retrovirus, the genetically modified T-cell or the pharmaceutical composition according to the present disclosure for manufacturing medicaments for treating diseases mediated by BCMA.

In one or more embodiments, the disease mediated by BCMA is multiple myeloma.

### DESCRIPTION OF DRAWINGS

FIG. 1 depicts a retrovirus expression vector of the BCMA-CAR (BCMA-41BBz) according to the disclosure. SP: signal peptide; VL: light chain variable region; Lk: linker (G₄S)₃; VH: heavy chain variable region; H: hinge region of CD8α; TM: transmembrane region of CD8.

FIG. 2 shows part of the peaks in the sequencing of the retrovirus expression vector of BCMA-CAR (BCMA-41BBz).

FIG. 3 depicts a retrovirus expression vector of the BCMA-tEGFR-CAR (BCMACAR-tEGFR) according to the disclosure. SP: signal peptide; VL: light chain variable region; Lk: linker (G₄S)₃; VH: heavy chain variable region; H: hinge region of CD8α; TM: transmembrane region of CD8; 2A: P2A peptide.

FIG. 4 shows part of the peaks in the sequencing of the retrovirus expression vector of pRetro-BCMA-tEGFR-CAR (BCMACAR-tEGFR).

FIG. 5 shows the flow cytometry of BCMA-tEGFR-CAR+ expression by T-cells infected with the retrovirus for 72 hours.

FIG. 6 shows the flow cytometry of BCMA expression by target cells.

FIG. 7 shows the CD107a expression by a 5-day preparation of BCMA-tEGFR-CART-cells incubated with the target cells for 5 hours.

FIG. 8 shows the INF-γ secretion by a 5-day preparation of BCMA-tEGFR-CART-cells incubated with the target cells for 5 hours.

FIG. 9 shows the killing of tumor cells by a 5-day preparation of BCMA-tEGFR-CART-cells after incubation with the target cells for 20 hours.

### DETAILED DESCRIPTION OF THE INVENTION

The present disclosure provides a BCMA-targeting chimeric antigen receptor (CAR). The CAR comprises the followings linked in sequence: an anti-BCMA single chain antibody, the hinge region of human CD8α, the transmembrane region of human CD8, the intracellular domain of human 41BB, the intracellular domain of human CD3ζ and optionally a fragment of EGFR comprising the extracellular domain III and the extracellular domain IV of the receptor.

A suitable anti-BCMA single chain antibody may be one derived from any of the anti-BCMA monoclonal antibodies known to a person in the art.

Optionally, the light chain variable region and the heavy chain variable region may be joined by a linker. Examples of these single chain antibodies include but are not limited to C11D5.3, J22.9. In some embodiments, the monoclonal antibody is the one of Clone C11D5.3. In some embodiments, the anti-BCMA single chain antibody has a light chain variable region having the sequence of amino acids 22-132 as set forth in SEQ ID NO:2. In some other embodiments, the anti-BCMA single chain antibody has a heavy chain variable region having the sequence of amino acids 148-264 as set forth in SEQ ID NO:2.

The hinge region of human CD8α useful in the present disclosure may have the sequence of amino acids 265-311 as set forth in SEQ ID NO:2.

The transmembrane region of human CD8 useful in the present disclosure may be any one of the human CD8 transmembrane sequences useful in CARs. In some embodiments, the transmembrane region of human CD8 has the sequence of amino acids 312-333 as set forth in SEQ ID NO:2.

The 41BB useful in the present disclosure may be any one of the 41BB molecules useful in CARs. In an illustrative example, the 41BB used in the present disclosure has the sequence of amino acids 334-381 as set forth in SEQ ID NO:2.

The intracellular domain of human CD3ζ useful in the present disclosure may any one of the intracellular domains of human CD3ζ useful in CARs. In some embodiments, the intracellular domain of human CD3ζ has the sequence of amino acids 382-492 as set forth in SEQ ID NO:2.

These components of the fusion protein of the present disclosure, say the light chain variable region and the heavy chain variable region of the anti-BCMA single chain antibody, the hinge region of human CD8α, the transmembrane region of human CD8, 41BB, the intracellular domain of human CD3ζ etc., may be linked one to another directly or via linkers. The linkers may be any of those used in antibodies, like those comprising G and S. Usually, linkers comprise repeats of one or more motifs. Examples of the motif include GGGS, GGGGS, SSSSG, GSGSA and GGSGG. Preferably, the motifs are adjacent one another in a linker, without in-between amino acid residues. The linker may comprise 1, 2, 3, 4 or 5 repeats of a motif. The linker may be 3-25 amino acid residues, for example, 3-15, 5-15, 10-20 amino acid residues in length. In some embodiments, the linker is a poly(glycine) linker. There is no limit on the number of glycine residues in the linker, while the number is usually in the range of 2-20, for example, 2-15, 2-10 and 2-8. Apart from glycine and serine, the linker may further comprises some additional amino acid residues, like alanine (A), leucine (L), threonine (T), glutamic acid (E), phenylalanine (F), arginine (R), glutamine (Q), etc. Examples of the linker are those of the amino acid sequences as set forth in SEQ ID NOs:7-12. In some embodiments, in the anti-BCMA single chain antibody, the light chain variable region and the heavy chain variable region are linked via (GGGGS)ₙ, wherein n is an integer from 1 to 5.

In some embodiments, the CAR of the present disclosure has the sequence of amino acids 22-492 as set forth in SEQ ID NO:2 or the sequence of amino acids 1-492 as set forth in SEQ ID NO:2. In some embodiments, the CAR of the present disclosure may further comprises in its amino acid sequence a fragment of EGFR comprising the extracellular domain III and the extracellular domain IV of the receptor, the signal peptide thereof and a linker.

It should be understood that due to restriction sites designed for cloning, the expressed amino acid sequence will include one or more irrelevant residues at the end(s), which will not interfere the activity of the sequence of interests. To construct a fusion protein, to increase recombinant expression, to enable spontaneous secretion to outside of the hosts or to facilitate purification, the protein may need to include some additional amino acids at the N-terminal, the C-terminal of some other part of the fusion protein as appropriate. For example, the additional amino acids include but are not limited to a linker peptide, a signal peptide, a leader, a terminal extension. Accordingly, the fusion protein (i.e., the CAR) of the present disclosure may further include at the N- or the C-terminal one or more polypeptide fragment(s) as protein tag(s). Any suitable tags are useful in the present disclosure. For example, the tag may be FLAG, HA, HA1, c-Myc, Poly-His, Poly-Arg, Strep-TagII, AU1, EE, T7, 4A6, E, B, gE and Ty1. These tags are useful in protein purification.

The present disclosure further includes variants of the CAR having the sequence of amino acids 24-495 as set forth in SEQ ID NO:2, the CAR having the sequence of amino acids 24-878 as set forth in SEQ ID NO:2, the CAR having the sequence of amino acids 1-495 as set forth in SEQ ID NO:2 or the CAR having the amino acid sequence of SEQ ID NO:2. The variant includes a amino acid sequence that has a sequence identity of at least 80%, preferably at least 85%, preferably at least 90%, preferably at least 95%, preferably at least 97% to the specified CAR and the same biological activity (e.g., of T-cell activation) as the specified CAR. Sequence identity can be calculated, for example, using BLASTp from NCBI.

Variants also include those having one or a plurality of mutation(s) (insertion, deletion or substitution) in the sequence of amino acids 22-492 as set forth in SEQ ID NO:2, the sequence of amino acids 22-874 as set forth in SEQ ID NO:2, the sequence of amino acids 1-492 as set forth in SEQ ID NO:2 or the amino acid sequence of SEQ ID NO:2 and remaining the biological activity of the CAR. The “plurality” normally refers to a number raging from 1 to 10, such as from 1 to 8, from 1 to 5 or from 1 to 3. The substitution is preferably a conservative one. For instance, conservative substitution between amino acids close or similar in property is known as will not change the effect of the protein or polypeptide. “Amino acids close or similar in property” include, for example, a family of amino acid residues having similar side chains. These families include, for example, amino acids with a basic side chain (e.g., lysine, arginine, histidine), amino acids with an acidic side chain (e.g., aspartic acid, glutamic acid), amino acids with an uncharged polar side chain (e.g., glycine, asparagine, glutamine, serine, threonine, tyrosine, cysteine), amino acids with a nonpolar side chain (e.g., alanine, valine, leucine, isoleucine valine, phenylalanine, methionine, tryptophan), amino acids with a β-branched side chain (e.g., threonine, proline, isoleucine) and amino acids with an aromatic side chain (e.g., tyrosine, phenylalanine, tryptophan, histidine). Accordingly, in the polypeptides according to the present disclosure, substitution with another member of the same family of side chain will not cause any substantive change in activity.

The present disclosure includes polynucleotide sequences encoding the fusion proteins according to the present disclosure. The polynucleotide sequences of the present disclosure may be in the form of DNA or RNA. The term “DNA” includes cDNA, genomic DNA or artificial synthetic DNA. The DNA may be a single-strand or a doubled-strand DNA. The DNA may be the coding strand or the non-coding strand. The present disclosure further includes degenerate variants of the polynucleotide sequence encoding the fusion protein, i.e., different nucleotide sequences that encode the same amino acid sequence.

The polynucleotide sequences according to the present disclosure can be prepared by PCR amplification. Specifically, the sequence can be amplified using primers designed according to the given nucleotide sequence (particularly the Open Reading Frames) and commercially available cDNA libraries or self-made cDNA libraries as templates. For longer sequences, two or more runs will be needed, and fragments from each run are then assembled into the correct sequence. For example, in some embodiments, the polynucleotide sequence encoding the fusion protein of the present disclosure has the sequence of nucleotides 64-1476 as set forth in SEQ ID NO:1 or the sequence of nucleotides 1-1476 as set forth in SEQ ID NO:1.

In some embodiments, the polynucleotide sequence of the present disclosure further comprises the nucleotide sequence encoding the fragment of EGFR.

Any EGRF known to a person in the art is useful in the present disclosure, which include, for example, a human-derived EGFR. EGFRs comprise at N-terminal extracellular domains I and II, extracellular domain III, extracellular domain IV, a transmembrane region, a juxtamembrane domain and a tyrosine kinase domain. In a preferred embodiment, used in the present disclosure is a truncated EGFR (“tEGFR”, AKA the “fragment of EGFR” in the present disclosure), particularly, a truncated EGFR lacking the intracellular region (juxtamembrane domain and tyrosine kinase domain). In some embodiments, the EGFR without intracellular region is further truncated to lack the extracellular domains I and II. Accordingly, in some embodiments, used in the present disclosure is a tEGFR comprising or consisting of the extracellular domain III, the extracellular domain IV and the transmembrane region of EGFR. In some embodiments, the tEGFR comprises or consists of the sequence of amino acids 310-646 of human EGFR, wherein the sequence of amino acids 310-480 corresponds to the extracellular domain III of human EGFR, the sequence of amino acids 481-620 corresponds to the extracellular domain IV of human EGFR, and the sequence of amino acids 621-646 corresponds to the transmembrane region of human EGFR. In some examples, the tEGFR comprises the extracellular domains III and IV having the sequence of amino acids 518-539 as set forth in SEQ ID NO:2.

The tEGFR may comprise ahead of the N-terminal a leader sequence to facilitate expression. In some embodiments, the present disclosure may use the signal peptide from the α chain of GM-CSF receptor (“GMCSFR”). In some embodiments, the signal peptide has the sequence of amino acids 522-543 as set forth in SEQ ID NO:2.

Additionally, a sequence encoding P2A polypeptide may be use to link the sequence encoding the signal peptide and the tEGFR to the sequence encoding the human CD3ζ intracellular domain of the present disclosure CAR. In one or more embodiments, the P2A peptide has the sequence of amino acids 493-521 as set forth in SEQ ID NO:2.

Accordingly, in some embodiments, the polynucleotide sequence according to the present disclosure comprises a sequence encoding the CAR according to the present disclosure, a sequence encoding P2A polypeptide, a sequence encoding the signal peptide from the α chain of GM-CSF receptor and a sequence encoding the tEGFR. In some embodiments, the polynucleotide sequence according to the present disclosure has the sequence of nucleotides 64-2628 as set forth in SEQ ID NO:1, or the nucleotide sequence of SEQ ID NO:1.

The present disclosure further includes a nucleic acid construct, which comprises the polynucleotide sequence according to the present disclosure operably linked to one or more regulatory sequence(s). The polynucleotide sequence according to the present disclosure can be manipulated in various ways to ensure a successful expression of the fusion protein (CAR and/or tEGFR). Before being inserted into a vector, the nucleic acid construct may be adaptively processed according to the selected expression vector. The recombinant DNA techniques useful to modify polynucleotide sequences are already known.

The regulatory sequence may be a proper promoter sequence. The promoter sequence is usually operably linked to the coding sequence of the protein to be expressed. The promoter may be any of the nucleotide sequences that exhibit transcription activity in the host, which includes mutated, truncated or hybrid promoters, and which may be obtained from the gene of an extracellular or intracellular polypeptide that is homologous or heterologous to the host. A regulatory sequence may also be a transcription terminator sequence as appropriate, which terminates transcription upon recognition by the host cell. The terminator sequence is operably linked to the 3′-end of the nucleotide sequence encoding the polypeptide. Any terminator functional in a selected host is useful in the present disclosure. The regulatory sequence may also be a leader sequence as appropriate, which is a untranslated region of mRNA important for translation in the host cell. The leader sequence is operably linked to the 5′-end of the nucleotide sequence encoding the polypeptide. Any leader sequence functional in a selected host is useful in the present disclosure.

In some embodiments, the nucleic acid construct is a vector. Typically, the polynucleotide sequence according to the present disclosure is operably link to the promoter, and the construct is incorporated into an expression vector to obtain an effective expression of the polynucleotide sequence according to the present disclosure. The vector may be one suitable for replication in and integration into an eukaryotic cell. Typically, a cloning vector comprises a transcription terminator, a translation terminator, an initiation region and a promoter to modulate the desired expression of a nucleic acid sequence.

The polynucleotide sequence according to the present disclosure may be cloned into various types of vectors. For example, it can be cloned into a plasmid, a phagemid, a phage derivative, an animal virus or a cosmid. Further, the vector may be an expression vector. The expression vector may be delivered into the cell in form of a virus vector. Viral vector technology is already known and has been described in, for example, Molecular Cloning: A Laboratory Manual, Cold Spring Harbor Laboratory, Sambrook et al., 2001, New York) and many other virology and molecular biology manuals. Viruses that are useful as vectors include but are not limited to retrovirus, adenovirus, adeno-associated virus, herpes virus and lentivirus.

Typically, a suitable vector comprises a replication origin, promoter sequence, convenient restriction site and one or more selectable markers that are functional in at least one organism (e.g., WO 01/96584; WO01/29058; and U.S. Pat. No. 6,326,193).

For instance, in some embodiments, used in the present disclosure is a retrovirus vector, which comprises an origin of replication, a 3′LTR, a 5′LTR, the polynucleotide sequence according to the present disclosure, and optionally a selectable marker.

An example of suitable promoters is the immediate early promoter of cytomegalovirus (CMV). This is a strong constitutive promoter that is capable of driving high expression of whatever polynucleotide sequence operably linked to it. Another example of suitable promoters is extension growth factor-1α (EF-1α). Still, other constitutive promoter sequences may also be used, including but not limited to: simian virus 40 (SV40) early promoter, mouse mammary tumor virus (MMTV), human immunodeficiency virus (HIV) long terminal repeat (LTR) promoter, MoMuLV promoter, avian leukosis virus promoter, EB virus immediate early promoter, Rous sarcoma virus promoter, and human gene promoter, such as actin promoter, myosin promoter, heme promoter and creatine kinase promoter. Further, also contemplated are inducible promoters. Inducible promoters provide a molecular switch, which can turn on the expression of the polynucleotide sequence operably linked to the inducible promoter as desired, and turn off when the expression is not desired. Examples of inducible promoter include but are not limited to metallothionein promoter, glucocorticoid promoter, progesterone promoter and tetracycline promoter.

For assessment of expression of the CAR polypeptide or part of it, the expression vector to be introduced into cells may further comprise a selectable marker gene or a reporter gene or both, such that expression cells can be identified or selected from the cell population transfected or infected with the virus vector. In some other aspects, the selectable marker may be carried on a separate DNA sequence for use in co-transfection. Both the selectable marker and the reporter genes may be flanked by one or more regulatory sequences for expression in host cells. Useful selectable markers include for example antibiotics resistance genes, like neo, etc.

Reporter genes are used to identify potentially transfected cells and to assess functionality of the regulatory sequences. After DNA being transferred into the recipient cells, the reporter gene may be detected at an appropriate time point. Suitable reporter genes may include those encoding luciferase, β-galactosidase, chloramphenicol acetyltransferase, secreted alkaline phosphatase or green fluorescent protein. Suitable expression systems are already known and can be prepared using existing techniques or commercially obtained.

Methods for transferring genes into and for expressing genes in cells are already known. Vectors can be conveniently delivered into host cells, like mammalian, bacterial, yeast or insect cells, using various methods as known to a person in the art. For instance, an expression vector may be transferred into the host cell using a physical, chemical or biological means.

Physical methods of transferring polynucleotides into host cells include calcium phosphate precipitation, lipofection, particle bombardment, microinjection, electroporation, etc. Biological methods of transferring polynucleotides into host cells involve DNA and RNA vectors. Chemical methods of transferring polynucleotides into host cells involve colloidal dispersion systems, such as macromolecular complexes, nanocapsules, microspheres and beads; and lipid-based systems, including oil in water emulsion, micelle, mixed micelles and liposomes.

Biological methods of transferring polynucleotides into host cells involve virus vectors, especially retrovirus vector, which has been widely used in gene integration into mammalian cells, like human cells. Additional virus vectors may be those derived from lentivirus, poxvirus, simple herpes virus I, adenovirus, adeno-associated virus, etc. Many virus-based systems have been developed for use in gene transfer into mammalian cells. For example, retroviruses provide a convenient platform for gene transfer systems. A selected gene may be inserted into a vector and then packaged into a retrovirus particle using techniques as known to a person in the art. The recombinant virus may then be isolated and transferred into cells from a subject in vivo or ex vivo. There are many retrovirus systems as known to a person in the art. In some examples, adenovirus vectors may be used. There are many adenovirus vectors as known to a person in the art. In one embodiment, a lentivirus vector is used.

Accordingly, in some embodiments, the present disclosure further provides a retrovirus useful in T-cell activation, wherein the virus comprises the retrovirus vector according to the present disclosure and corresponding package genes, like gag, pol and vsvg.

T-cells useful in the present disclosure can be those of any origins and of any types. For example, T-cells may be those from PBMC from a patient having B-cell malignant tumor.

In some embodiments, before use, the obtained T-cells may first be stimulated using an appropriate amount (such as 30-80 ng/ml, e.g., 50 ng/ml) of an anti-CD3 antibody, and then cultured in a medium supplemented with an appropriate amount (such as 30-80 IU/ml, e.g., 50 IU/ml) of IL2.

Accordingly, in some embodiments, the present disclosure provides a genetically modified T-cell, which comprises the polynucleotide sequence according to the present disclosure or the retrovirus vector according to the present disclosure, or is infected with the retrovirus vector according to the present disclosure, or is prepared by the method according to the present disclosure, or stably expresses the fusion protein and optionally the tEGFR according to the present disclosure.

The CAR-T-cells according to the present disclosure may undergo robust in vivo T-cell expansion, sustain in blood and bone marrow for a prolonged time period, and form specific memory T-cells. Without being bound to any particular theory, after encountering and depleting the target cells expressing the antigen substitute, the CAR-T-cells according to the present disclosure can differentiate into the central memory status in vivo.

The present disclosure further includes a kind of cell therapy, wherein T-cells are genetically modified to express the CAR and optionally the tEGFR according to the present disclosure, and the CAR-T-cells are infused into a recipient in need of such a therapy. The infused cells kill tumor cells in the recipient. Unlike antibody therapy, CAR-T-cells are capable of in vivo replication and production, which leads to a long-term sustained control of tumor.

The CAR-T-cells-mediated anti-tumor immune response may be an active or a passive one. Additionally, the CAR-mediated immune response may be part of an adoptive immunotherapy, wherein the CAR-T-cells induce an immune response with a specificity defined by the antigen-binding part of the CAR.

Accordingly, the diseases that can be treated using the CAR, the sequence encoding same, the nucleic acid construct, the expression vector, the virus and the CAR-T-cells according to the present disclosure are preferably diseases mediated by BCMA.

The CAR-modified T-cells according to the present disclosure may be used alone or in form of a pharmaceutical composition and in combination with a diluent and/or other components like relevant cytokine(s) or cell population(s). Briefly, the pharmaceutical composition according to the present disclosure may comprise the CAR-T-cells according to the present disclosure in combination with one or more pharmaceutically or physiologically acceptable carrier(s), diluent(s) or excipient(s). The composition may comprise a buffer solution, such as a neutral buffered saline, sulfate buffered saline, etc; a carbohydrate, such as glucose, mannose, sucrose or dextran, mannitol; a protein; a polypeptide or an amino acid, such as glycine; an antioxidant; a chelator, such as EDTA or glutathione; an adjuvant (e.g., aluminum hydroxide); and a preservative.

The pharmaceutical composition according to the present disclosure may be administered in a manner as appropriate for the disease that is to be treated or prevented. The amount and frequency of administration will be determined by known factors, like the medical condition of the patient and the classification and severity of the disease.

When referring to “an immunologically effective amount”, “an anti-tumor effective amount”, “a tumor-inhibition effective amount” or “a therapeutically effective amount”, the exact amount at which the composition according to the present disclosure is to be administered will be determined by a physician on an individual basis with considerations including patient's (subject's) age, body weight, tumor size, degree of invasion or metastasis. Typically, the pharmaceutical composition comprising the T-cell may be administered at an dosage ranging from 10⁴ to 10⁹ cells/kg bodyweight, preferably 10⁵ to 10⁶ cells/kg bodyweight. The T-cell composition may also be administered multiple times by repeating the specified dosage. The cells may be administered using conventional infusion techniques as known in immunotherapy (see for example, Rosenberg et al., New Eng. J. of Med. 319:1676, 1988). The optimal dosage and regimen for a particular patient may be conveniently determined by monitoring the patient's signs of disease and making adjustment accordingly.

The composition may be administered in any way as convenient, like aerosol, injection, swallowing, infusion, implantation or transplantation. The composition may be administered to a patient subcutaneously, intradermally, intratumorally, intraductally, intraspinally, intramuscularly, intravenously or intraperitoneally. In one example, the T-cell composition according to the present disclosure is administered via intradermal or subcutaneous injection. In another example, the T-cell composition is preferably administered via intravenous injection. The T-cell composition may be directly injected into the tumor, lymph nodes or sites of infection.

In some examples according to the present disclosure, the CAR-T-cells or the composition may be supplied in combination with an additional therapy. The additional therapy may include but is not limited to chemotherapy, radiation and immunosuppressants. For instance, the additional therapy may be any of the radio- or chemo-therapies known as useful in diseases mediated by BCMA.

The term “anti-tumor effect”, as used herein, refers to a biological effect that is characterized in decreased tumor size, reduced number of tumor cells, reduced metastasis, increased life expectancy or improvement in any physiological symptoms associated with cancer.

The terms “patient”, “subject”, “individual” are used herein as exchangeable and all refer to a living organism, like an mammalian, in which the immune response can be induced. Examples include but are not limited to human beings, dogs, cats, mice, rats and corresponding transgenic species.

The present disclosure, utilizing the gene sequences of anti-BCMA antibodies (specifically, the scFV derived from Clone C11D5.3) and the sequences of the hinge region of human CD8α, the transmembrane region of human CD8, the intracellular domain of human 41BB and the intracellular domain of human CD3ζ from NCBI GenBank, synthesized the whole gene of the chimeric antigen receptor “anti-BCMA scFv-CD8 hinge-CD8TM-41BB-CD3ζ” and the whole gene of “anti-BCMA scFv-CD8 hinge-CD8TM-41BB-CD3ζ-GMCSFR leader-tEGFR”, which is then incorporated into a retrovirus vector. The recombinant plasmids are packaged into viruses in 293T-cells. The resultant viruses are then used to infect T-cells to express the chimeric antigen receptor on the cells. According to the present disclosure, the chimeric antigen receptor-genetically modified T lymphocytes are transformed using a retrovirus-based process, which advantageously provides inter alia a high efficiency of transformation, a stable expression of the exogenous gene and a shortened period of in vitro culturing before reaching a clinical-grade number of T lymphocytes. The transferred nucleic acid is transcribed and expressed on surface of the transgenic T lymphocytes. The CAR-T-cells prepared according to the present disclosure provides a strong killing (>70%) of the specific tumor cells at an effector-to-target ratio of 10:1. Further, when the CAR according to the present disclosure carries the tEGFR assembly, the assembly forms a spatial configuration allowing tightly binding to Cetuximab (an approved anti-EGFR monoclonal antibody drug) to provide a surface marker and meanwhile a means for in vivo tracing of the T-cells (e.g., by flow cytometry and immunohistochemical assays). It may also allow a depletion by Cetuximab. That is, Cetuximab may be added in case the effect of the CAR is unwanted, and this provides a safe control of the action of CAR-T-cells in vivo. Accordingly, the CAR according to the present disclosure further allows in vivo tracing and a safety cut-off.

The inventions will be described in further detail by reference to the following experimental examples. These examples are provided for illustrative purposes only and are not intended to be limiting unless otherwise specified. The inventions should never be construed as being limited to these following examples, but intended to include any and all variations that are obvious from the teachings provided herein. The methods and reagents used in the examples are those regular ones, unless otherwise indicated.

### Example 1: Gene Sequence of BCMA-scFv-CD8α-CD28-41BB-CD3

Gene sequences of the hinge region of human CD8α, the transmembrane region of human CD8α, the intracellular domain of 41BB and the intracellular domain of human CD3ζ were obtained from the database on the website of NCBI, and the anti-BCMA single chain antibody is derived from Clone C11D5.3. These sequences were codon-optimized at http://sg.idtdna.com/site to ensure a better expression in human cells without change in the amino acid sequence encoded thereby.

Overlap-PCR was conducted to link the sequences in the order as specified: the gene of the anti-BCMA scFv, the gene of the hinge region of human CD8α, the gene of the transmembrane region of human CD8α, the gene of the intracellular domain of 41BB, and the gene of the intracellular domain of human CD3ζ, with distinct restriction sites introduced at each of the adjunctions between sequences, whereby to generate the complete sequence of the BCMA-CAR.

The nucleotide sequence of the CAR molecule was double digested with NotI (NEB) and EcoRI (NEB), and inserted into the retrovirus vector MSCV (Addgene) at the NotI-EcoRI site using T4 ligase (NEB). The vector was then transferred into the competent Escherichia coli. strain (DH5α).

The obtained recombinant plasmid was sequenced by Sangon Biotech (Shanghai) Co., Ltd. The result was aligned to the to-be-synthesized BCMA CAR sequence to confirm the correct sequence. The sequencing primers are:

forward:
(SEQ ID NO: 3)
AGCATCGTTCTGTGTTGTCTC;
reverse:
(SEQ ID NO: 4)
TGTTTGTCTTGTGGCAATACAC.

After being confirmed correct by sequencing, the plasmids were purified using the plasmid purification kit from Qiagen. The purified plasmids were transferred into 293T-cells using the calcium phosphate-method for retrovirus packaging.

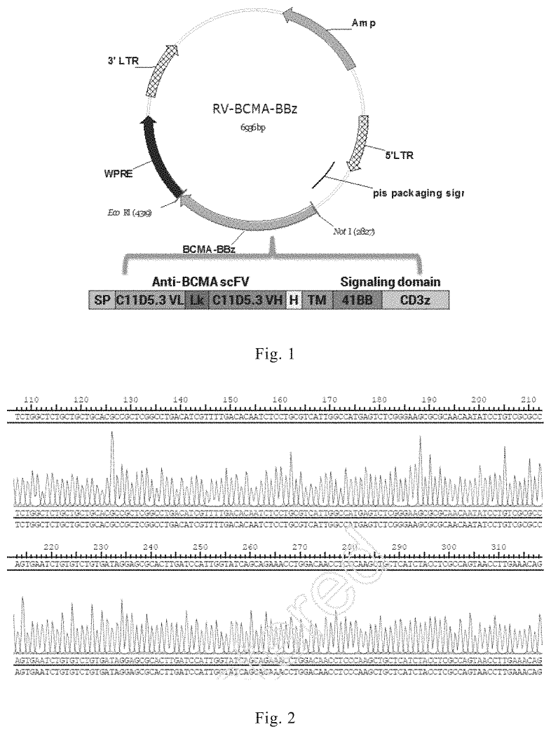

The plasmid constructed according to this example is schematically depicted in FIG. 1. FIG. 2 illustrates part of the peaks in the sequencing of this retrovirus expression plasmid.

### Example 2: Gene Sequence of BCMA CAR-GMCSFR Leader-tEGFR

Gene sequence of the extracellular domain of human EGFR was obtained from the database on the website of NCBI. The sequence was codon-optimized at http://sg.idtdna.com/site to ensure a better expression in human cells without change in the amino acid sequence encoded thereby

Overlap-PCR was conducted to link the sequences in the order as specified: the BCMA CAR of Example 1, the GMCSFR leader and the tEGFR, with distinct restriction sites introduced at each of the adjunctions between sequences, whereby to generate the complete sequence of the BCMA CAR-GMCSFR leader-tEGFR.

The nucleotide sequence of the CAR molecule was double digested with NotI (NEB) and EcoRI (NEB), and inserted into the retrovirus vector MSCV (Addgene) at the NotI-EcoRI site using T4 ligase (NEB). The vector was then transferred into the competent Escherichia coli. strain (DH5α).

The obtained recombinant plasmid was sequenced by Sangon Biotech (Shanghai) Co., Ltd. The result was aligned to the to-be-synthesized BCMA CAR-GMCSFR leader-tEGFR sequence to confirm the correct sequence. The sequencing primers are:

forward:
(SEQ ID NO: 5)
AGCATCGTTCTGTGTTGTCTC;
reverse:
(SEQ ID NO: 6)
TGTTTGTCTTGTGGCAATACAC.

After being confirmed correct by sequencing, the plasmids were purified using the plasmid purification kit from Qiagen. The purified plasmids were transferred into 293T-cells using the calcium phosphate-method for retrovirus packaging.

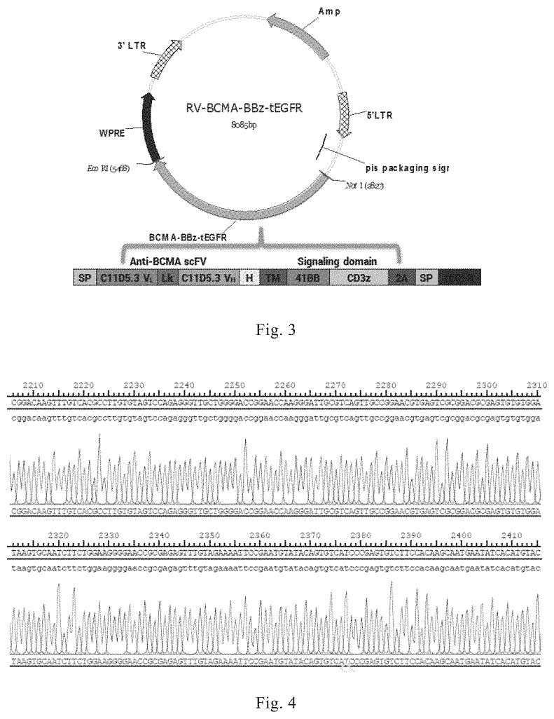

The plasmid constructed according to this example is schematically depicted in FIG. 3. FIG. 4 illustrates part of the peaks in the sequencing of this retrovirus expression plasmid.

### Example 3: Retrovirus Packaging

1. Day 1: The 293T-cells should be within the 20^{th} passage and not over-confluent. The cells were plated at 0.6×10⁶ cells/ml on a 10 cm dish containing 10 ml DMEM medium. The cells were mixed well until uniform, and cultured at 37° C. over night;

2. Day 2: Transfection was conducted on 293T-cells grown to 90% confluence (normally, about 14-18 hrs after plating); A plasmid complex was prepared, which comprised the plasmids each present at an amount of 12.5 μg based on the MSCV backbone, Gag-pol 10 μg, VSVg 6.25 μg, CaCl₂ 250 μl, H₂O 1 ml, in a total volume of 1.25 ml; Into a separate tube filled with HBS of the same volume as the plasmid complex, the plasmid complex was added with vortexing for 20 s; The obtained mixture was gently added along the vessel's wall into the petri dish of 293T, incubated at 37° C. for 4 hrs. Then the medium was removed and, after washing once with PBS, replaced with pre-warmed fresh medium.

3. Day 4: 48 hrs after transfection, the supernatant was collected and filtered through a 0.45 um filter, then divided into aliquots and stored at −80° C.; to the cells, pre-warmed fresh DMEM medium was supplemented.

### Example 4: Retrovirus Infection of Human T-Cells

1. CD3+T-cells were purified using Ficcol solution (Tian Jin Hao Yang Biological Manufacture Co., Ltd) and conditioned in X-VIVO (LONZA) medium supplemented with 5% AB serum till cell density of 1×10⁶/mL. The cells were inoculated at 1 ml/well onto a plate pre-treated with 50 ng/ml anti-human CD3 antibody (Beijing T&L Biotechnology Co. Ltd) and 50 ng/ml CD28 antibody (Beijing T&L Biotechnology Co. Ltd), followed by addition of 100 IU/ml IL-2 (Beijing SL Pharmaceutical Co. Ltd). After cultivation under stimulation for 48 hours, the cells were infected with the virus prepared in Example 3;

2. At the second day of the T-cell cultivation and activation, 24-well plates were prepared by coating with 250 μl/well of Retronectin (Takara) diluted in PBS to the final concentration of 15 μg/ml; The plates were kept in dark at 4° C. overnight for use;

3. Two days after the T-cell cultivation and activation, two coated 24-well plates were taken out, the coating solution was pipetted off, and HBSS supplemented with 2% BSA was added at 500 μl/well to block at room temperature for 30 min; Then, the blocking solution was pipetted off and the plates were washed twice with HBSS supplemented with 2.5% HEPES;

4. Each well was added with 2 ml virus solution/well of the virus solution prepared in Example 3, and the plate was then centrifuged at 2000 g, 32° C. for 2 hrs;

5. Supernatant being discarded, into each well on the 24-well plates, 1×10⁶ activated T-cells were added in a volume of 1 ml, wherein the medium is the T-cell culturing medium supplemented with 200 IU/ml of IL-2; The plates were centrifuged at 1000 g, 30° C. for 10 min;

6. After centrifugation, the plates were incubated in an incubator at 37° C., 5% CO₂;

7. 24 hrs after infection, the cell suspension was pipetted and centrifuged at 1200 rpm, 4° C. for 7 min;

8. Since the infection, cell density was measured every day; T-cell culturing medium supplemented with 100 IU/ml of IL-2 was added as appropriate to maintain T-cell density at around 5×10⁵/ml and to effect cell expansion;

Thereby, CAR T-cells infected with the retrovirus of Example 3 were obtained, which were respectively named “BCMA CAR T-cells” (expressing the BCMA CAR according to Example 1) and “BCMA-tEGFR CAR T-cells” (expressing the BCMA CAR and tEGFR according to Example 2).

### Example 5: Flow Cytometric Assays of Infected T-Lymphocytes for their Proportion and the Expression of Surface CAR Protein

The CAR-T-cells and the NT-cells (control) were respectively collected by centrifugation 72 hours after the infection according to Example 4. The cells were washed once with PBS and the supernatant was discarded. The cells were then exposed to corresponding antibodies in dark for 30 min, washed again with PBS, re-suspended and assayed via flow cytometry. CARP was detected using anti-mouse IgG F(ab′) antibody (Jackson Immunoresearch).

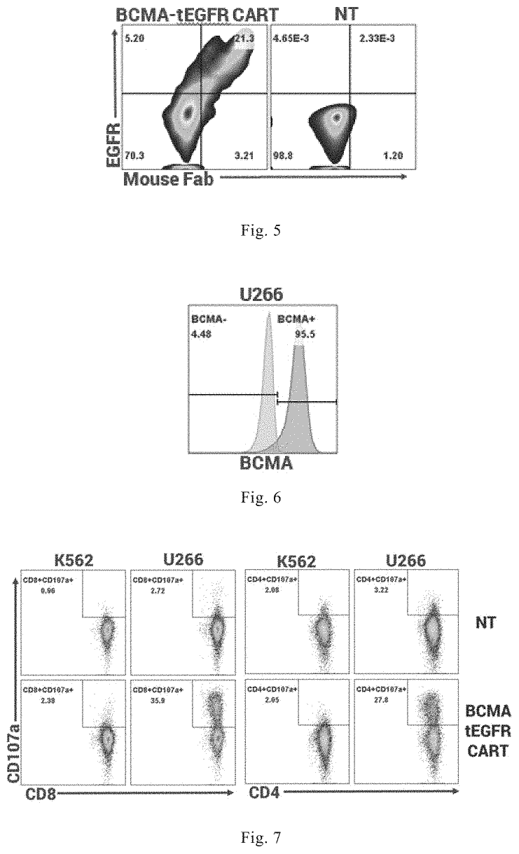

As exhibited in FIG. 5, in the T-cells 72 hours after infection using the retrovirus prepared in Example 3, the percentage of BCMA-tEGFR CAR+ expression was as high as 24.51%.

As exhibited in FIG. 6, BCMA percentage in target cells was detected using a BCMA antibody, and the percentage in U266 cells was 95.5%, which indicates that the target cell has a high expression of BCMA.

### Example 6: CD107a Expression by CAR-T-Cells Co-Incubated with Target Cells

1. Adding CART/NT-cell (2*10⁵ cells/well) and target cells (U266)/control (K562, 2*10⁵ cells/well) onto a V-bottom 96-well plate; Re-suspending in 100 ul X-VIVO complete medium free of IL-2; Adding BD GolgiStop (with monesin, 1 μl BD GolgiStop per 1 ml medium); Adding into each well 2 ul CD107a antibody (1:50), followed by incubation at 37° C. for 4 hours, then collecting the cells;

2. Removing supernatant from the sample by centrifugation, washing the cells once with PBS, centrifuging at 400 g, 4° C., 5 min; Discarding supernatant, adding into each tube an appropriate amount of antibodies specific for surface molecules CD3, CD4 and CD8 respectively; Re-suspending into 100 ul, followed by incubation on ice in dark for 30 min.

3. Washing the cells in each tube once with 3 mL PBS, then centrifuging at 400 g, 5 min; Carefully pipetting off the supernatant;

4. Re-suspending in an appropriate volume of PBS, followed by flow cytometry for CD107a.

As exhibited in FIG. 7, BCMA-tEGFR CART-cells were observed with a CD107a secretion percentage of 35.9% in CD8-positive U266 cells, and BCMA-tEGFR cells with a CD107a secretion percentage of 27.8% in CD4-positive U266 cells.

### Example 7: INF-γ Secretion by CAR-T-Cell Co-Incubated with Target Cells

1. Prepared CAR-T-cells were re-suspended in the Lonza medium, wherein cell density was adjusted to 1×10⁶/mL.

2. The test groups comprised in each well 2×10⁵ target cells (U266) or negative control cells (K562), 2×10⁵ CAR-T-cells and 200 μl Lonza medium free of IL-2. The mixture was added onto a 96-well plate. BD GolgiPlug (with BFA, 1 μl BD GolgiPlug/1 ml cell culture) was added at the same time and mixed well. The mixture was then incubated at 37° C. for 5-6 hours. The cells were collected as the test group.

3. Each tube was washed once with 1 mL PBS, then centrifuged at 300 g for 5 min. Supernatant was carefully pipetted off.

4. After washing with PBS, Fixation/Permeabilization solution was added at 250 μl/EP tube followed by incubation at 4° C. for 20 min to fix the cells and to disrupt cell membrane. The cells were washed twice with 1×BD Perm/Wash™ buffer, 1 mL each time.

5. The cells were stained for intracellular cytokines: certain amount of IFN-γ cytokine fluorescent antibody or negative control was diluted in BD Perm/Wash™ buffer to 50 μl. Cells after fixation and membrane disruption were re-suspended in this diluted antibody solution, and incubated at 4° C. in dark for 30 min, washed twice with 1×BD Perm/Wash™ buffer 1 mL/time, and re-suspended in PBS.

6. Detection was carried out by flow cytometry assay.

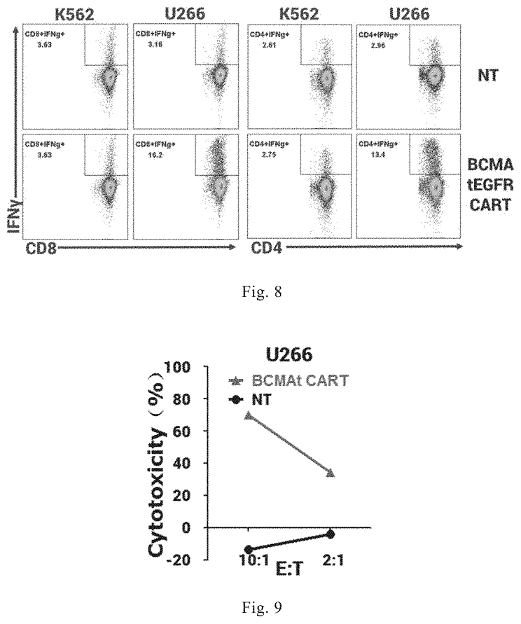

As exhibited in FIG. 8, BCMA-tEGFR CART-cells were observed with a INF-γ secretion percentage of 16.2% in CD8-positive U266 cells, and BCMA-tEGFR cells with a INF-γ secretion percentage of 13.4% in CD4-positive U266 cells.

### Example 8: Tumor-Specific Killing by CAR-T-Cells Incubated with Target Cells

1. K562 cells (BCMA target protein free, serving as the negative control relative to the target cells) were re-suspended in serum-free medium (1640), with cell concentration adjusted to 1×10⁶/ml, followed by addition of Fluorescent dye BMQC (2,3,6,7-tetrahydro-9-bromomethyl-1H, 5H-quinolizino(9,1-gh)coumarin) to the final concentration of 5 μM.

2. After mixed to uniform, the mixture was incubated at 37° C. for 30 min.

3. After centrifugation at room temperature, 1500 rpm for 5 min and the supernatant being discarded, the cells were re-suspended in the cytotoxicity medium (phenol red-free 1640+5% AB serum) and incubated at 37° C. for 60 min.

4. The cells were washed twice with fresh cytotoxicity medium and re-suspended in fresh cytotoxicity medium to 1×10⁶ cells/ml.

5. U266 cells (BCMA target protein positive, serving as target cells) were re-suspended in PBS supplemented with 0.1% BSA, and cell concentration was adjusted to 1×10⁶ cells/ml.

6. Fluorescent dye CFSE (carboxyfluorescein diacetate succinimidyl ester) was added to the final concentration of 1 μM.

7. After mixed to uniform, the mixture was incubated at 37° C. for 10 min.

8. At the end of incubation, an equal volume of FBS was added and incubated under room temperature for 2 min to end the labeling reaction.

9. The cells were washed and re-suspended in the fresh cytotoxicity medium to 1×10⁶ cell s/ml.

9. The effector T-cells were washed and re-suspended in the cytotoxicity medium and the concentration was adjusted to 5×10⁶ cells/ml.

10. In all experiments, cytotoxicity of the CAR-T-cells was compared with cytotoxicity of the uninfected negative control effector T-cells (NT-cells) that came from the same patient.

11. The CAR-T cells and the NT cells were incubated at the effector:target cell ratios=10:1, 2:1 in 5 ml sterile tubes (BD Biosciences), two duplicates for each group. In each of the groups of co-incubation, the target cells were U266 cells (50,000 cells, 50 μl), and the negative control cells were K562 cells (50,000 cells, 50 μl). At the same time, another group is designed to merely comprise U266 target cells and K562 negative control cells.

12. The cells were co-incubated at 37° C. for 5 hrs.

13. At the end of incubation, 7-AAD (7-aminoactinomycin D) was added according to instruction immediately after the cells were washed with PBS, and then incubated on ice for 30 min.

14. The cells were directly loaded onto the flow cytometer, and the data were analyzed using Flow Jo.

15. The analysis was gated by 7AAD-negative living cells to detect the percentage of living U266 target cells and the percentage of living negative control K562 cells after co-incubation of the T-cells and the target cells.

16. For each group of co-incubation of the T-cells and the target cells:

17. % cell killed by cytotoxicity=100−calibrated target cell survival %, i.e., (the number of living U266 cells in absence of the effector cells−the number of living U266 cells in presence of the effector cells)/the number of living K562 cells.

As exhibited in FIG. 9, at the effector:target ratio of 10:1, the BCMA-tEGFR CART-cells killed 70% of U266 cells.

What is claimed is:

1. A polynucleotide sequence selected from the group consisting of:
(1) a polynucleotide sequence comprising the followings linked in sequence: a sequence encoding an anti-BCMA single chain antibody, a sequence encoding the hinge region of human CD8α, a sequence encoding the transmembrane region of human CD8, a sequence encoding the intracellular domain of human 41BB, a sequence encoding the intracellular domain of human CD3ζ and optionally a sequence encoding a fragment of EGFR comprising the extracellular domain III and the extracellular domain IV; and
(2) a complementary sequence of the polynucleotide sequence of (1).

2. The polynucleotide sequence of claim 1, characterized in that a sequence encoding a signal peptide lies ahead of said sequence encoding the anti-BCMA single chain and has the sequence of nucleotides 1-63 as set forth in SEQ ID NO:1; and/or
the sequence encoding the light chain variable region of said anti-BCMA single chain antibody has the sequence of nucleotides 64-396 as set forth in SEQ ID NO:1; and/or
the sequence encoding the heavy chain variable region of said anti-BCMA single chain antibody has the sequence of nucleotides 442-792 as set forth in SEQ ID NO:1; and/or
the sequence encoding said hinge region of human CD8α has the sequence of nucleotides 793-933 as set forth in SEQ ID NO:1; and/or
the sequence encoding said transmembrane region of human CD8 has the sequence of nucleotides 934-999 as set forth in SEQ ID NO:1; and/or
the sequence encoding said intracellular domain of human 41BB has the sequence of nucleotides 1000-1143 as set forth in SEQ ID NO:1; and/or
the sequence encoding said intracellular domain of human CD3ζ has the sequence of nucleotides 1144-1476 as set forth in SEQ ID NO:1; and/or
a sequence encoding a linker between the signal peptide of the α chain of GM-CSF receptor and said intracellular domain of human CD3ζ has the sequence of nucleotides 1477-1554 as set forth in SEQ ID NO:1;
the sequence encoding the signal peptide of the α chain of GM-CSF receptor has the sequence of nucleotides 1555-1634 as set forth in SEQ ID NO:1; and/or
the sequence encoding said fragment of EGFR has the sequence of nucleotides 1635-2628 as set forth in SEQ ID NO:1; or
said polynucleotide sequence encodes the sequence of amino acids 24-495 as set forth in SEQ ID NO:2, or encodes the sequence of amino acids 24-878 as set forth in SEQ ID NO:2, or encodes the amino acid sequence of SEQ ID NO:2; or
said polynucleotide sequence comprises or consists of the nucleotide sequence of SEQ ID NO:1, the sequence of nucleotides 1-1476 as set forth in SEQ ID NO:1, the sequence of nucleotides 64-1476 as set forth in SEQ ID NO:1, or the sequence of nucleotides 64-2628 as set forth in SEQ ID NO:1.

3. The polynucleotide sequence of claim 1, a fusion protein selected from the group consisting of:
(1) a fusion protein comprising the followings linked in sequence: an anti-BCMA single chain antibody, the hinge region of human CD8α, the transmembrane region of human CD8, the intracellular domain of human 41BB and the intracellular domain of human CD3ζ as well as optionally a fragment of EGFR comprising the extracellular domain III and the extracellular domain IV; and
(2) a fusion protein derived from (1), comprising one or more substitution(s), deletion(s) or addition(s) in the amino acid sequence of (1) while retaining the activity of T-cell activation;
wherein, said anti-BCMA single chain antibody is preferably the anti-BCMA monoclonal antibody C11D5.3.

4. The polynucleotide sequence of claim 3, wherein, said fusion protein comprises one or more of the following features:
said fusion protein further comprises a signal peptide N-terminal to said anti-BCMA single chain antibody, wherein said signal peptide preferably has the sequence of amino acids 1-21 as set forth in SEQ ID NO:2;
the light chain variable region of said anti-BCMA single chain antibody has the sequence of amino acids 22-132 as set forth in SEQ ID NO:1;
the heavy chain variable region of said anti-BCMA single chain antibody has the sequence of amino acids 148-264 as set forth in SEQ ID NO:1;
said hinge region of human CD8α has the sequence of amino acids 265-311 as set forth in SEQ ID NO:1;
said transmembrane region of human CD8 has the sequence of amino acids 312-333 as set forth in SEQ ID NO:1;
said intracellular domain of human 41BB has the sequence of amino acids 334-381 as set forth in SEQ ID NO:1;
said intracellular domain of human CD3ζ has the sequence of amino acids 382-492 as set forth in SEQ ID NO:1; and
said fragment of EGFR comprises or consists of the extracellular domain III, the extracellular domain IV and the transmembrane region of EGFR; preferably, said fragment comprises or consists of the sequence of amino acids 310-646 of human EGFR; more preferably, said fragment has the sequence of amino acids 518-539 as set forth in SEQ ID NO:1;
and preferably, said fusion protein further comprises the signal peptide of the α chain of GM-CSF receptor, wherein said signal peptide of the α chain of GM-CSF receptor is positioned N-terminal to said fragment of EGFR; preferably, said signal peptide of the α chain of GM-CSF receptor has the sequence of amino acids 522-543 as set forth in SEQ ID NO:2; and preferably, said fusion protein further comprise a linker between said signal peptide of the α chain of GM-CSF receptor and said intracellular domain of human CD3ζ, wherein said linker preferably has the sequence of amino acids 493-517 as set forth in SEQ ID NO:2; and
preferably, said fusion protein has the sequence of amino acids 22-492 as set forth in SEQ ID NO:2, or the sequence of amino acids 22-646 as set forth in SEQ ID NO:2, or the sequence of amino acids 1-492 as set forth in SEQ ID NO:2, or the amino sequence of SEQ ID NO:2.

5. The polynucleotide sequence of claim 1, wherein a nucleic acid construct, comprising the polynucleotide sequence according to any one of claims 1 to 2;
preferably, said nucleic acid construct is a vector;
more preferably, said nucleic acid construct is a retrovirus vector comprising an origin of replication, a 3′-LTR, a 5′-LTR and the polynucleotide sequence according to claim 1 or 2.

6. The polynucleotide sequence of claim 5, a retrovirus, comprising the nucleic acid construct according to claim 5, preferably the vector and more preferably the retrovirus vector.

7. The polynucleotide sequence of claim 1, a genetically modified T-cell or a pharmaceutical composition comprising said genetically modified T-cell, wherein, said cell comprises the polynucleotide sequence according to claim 1 or the nucleic acid construct according to claim 5, or is infected with the retrovirus according to claim 6, or stably expresses the fusion protein and optionally the fragment of EGFR comprising the extracellular domain III and the extracellular domain IV according to claim 3

8. Use of the polynucleotide sequence of claim 1, the fusion protein of claim 3 or 4, the nucleic acid construct of claim 5 or the retrovirus of claim 6 for producing activated T-cells.

9. Use of the polynucleotide sequence according to claim 1, the fusion protein according to claim 3 or 4, the nucleic acid construct according to claim 5, the retrovirus according to claim 6 or the genetically modified T-cell or the pharmaceutical composition according to claim 7 for manufacturing a medicament for treating a disease mediated by BCMA;
preferably, said disease mediated by BCMA is multiple myeloma.
